(19) United States
(12) Patent Application Publication
(10) Pub. No.: US 2008/0279783 A1
(43) Pub. Date: Nov. 13, 2008

(54) ISOLATED NANOCAPSULE POPULATIONS AND SURFACTANT-STABILIZED MICROCAPSULES AND NANOCAPSULES FOR DIAGNOSTIC IMAGING AND DRUG DELIVERY AND METHODS FOR THEIR PRODUCTION

(75) Inventors: Margaret A. Wheatley, Media, PA (US); Brian E. Oeffinger, Mohnton, PA (US); Nikhil Dhoot, Wappingers Falls, NY (US); Justin Lathia, Elizabethtown, PA (US)

Correspondence Address: DRINKER BIDDLE & REATH LLP, One Logan Square, 18th & Cherry Streets, Philadelphia, PA, 19103-6996, US

(73) Assignee: Drexel University, Philadelphia, PA (US)

(21) Appl. No.: 11/849,759

(22) Filed: Sep. 4, 2007

Related U.S. Application Data
(62) Division of application No. 10/805,703, filed on Mar. 22, 2004, now abandoned.
(60) Provisional application No. 60/456,666, filed on Mar. 20, 2003.

(51) Int. Cl. A61K 49/22; A61K 9/50

(52) U.S. Cl. 424/9.52; 424/490

(57) ABSTRACT

A method for producing surfactant-stabilized microcapsules or nanocapsules including the steps of (a) preparing a suspension comprising a non-ionic sorbitan detergent and a salt in phosphate buffered saline; (b) adding to the suspension a nonionic polyoxyethylenesorbitan detergent to produce a solution; (c) heating while stirring the solution of step (b) to 55±5° C. and maintaining the temperature of the solution at 55±5° C. for several minutes; (d) allowing the solution to cool to room temperature; (e) autoclaving the solution; (f) creating surfactant-stabilized microbubbles and nanobubbles in the solution; and (g) collecting surfactant-stabilized nanocapsules and microcapsules formed from the microbubbles and nanobubbles.

### STATEMENT REGARDING FEDERALLY SPONSORED RESEARCH OR DEVELOPMENT

This invention was supported in part by funds from the U.S. government (NIH Grant Nos. HL052901 and CA52823). The U.S. government may therefore have certain rights in the invention.

### BACKGROUND OF THE INVENTION

1. Field of the Invention

Methods for isolating nanobubbles or nanocapsules from mixed populations of bubbles via separation into a lower layer as compared to large bubbles are described. Surfactant-stabilized microcapsules and/or nanocapsules for diagnostic imaging and drug delivery and methods for their production are also described. The present invention also relates to methods for production of a microbubble and/or nanobubble surfactant-based ultrasound contrast agent composed of a surfactant shell which can be modified to be loaded with bioactive compounds as well as a targeting moiety. In addition, the present invention provides methods for delivery of these nanocapsules alone or in combination with other agents including, but not limited to free drug, genetic material, non-echogenic capsules with or without drug payload, or combinations thereof. Methods are also provided for facilitating or enhancing delivery of nanocapsules to a selected tissue or tissues via vasculature and extravascular spaces too narrow for access with larger microcapsules, e.g., leaky tumor vasculature, using ultrasonic waves to force the nanocapsules through gaps in the vasculature and extravascular spaces by mechanisms including, but not limited to, cavitation and microstreaming.

2. Description of Related Art

Ultrasound contrast agents are used routinely in medical diagnostic, as well as industrial, ultrasound. For medical diagnostic purposes, contrast agents are usually gas bubbles, which derive their contrast properties from the large acoustic impedance mismatch between blood and the gas contained therein. Important parameters for the contrast agent include particle size, imaging frequency, density, compressibility, particle behavior (surface tension, internal pressure, bubble-like qualities), and biodistribution and tolerance.

Gas-filled particles are by far the best reflectors. Various bubble-based suspensions with diameters in the 1 to 15 micron range have been developed for use as ultrasound contrast agents. Bubbles of these dimensions have resonance frequencies in the diagnostic ultrasonic range, thus improving their backscatter enhancement capabilities. Sonication has been found to be a reliable and reproducible technique for preparing standardized echo contrast agent solutions containing uniformly small microbubbles. Bubbles generated with this technique typically range in size from 1 to 15 microns in diameter with a mean bubble diameter of 6 microns (Keller et al. 1986. J. Ultrasound Med. 5:493-498). However, the durability of these bubbles in the blood stream has been found to be limited and research continues into new methods for production of microbubbles. Research has also focused on production of hollow microparticles for use as contrast agents wherein the microparticle can be filled with gas and used in ultrasound imaging. These hollow microparticles, however, also have uses as drug delivery agents when associated with drug products. These hollow microparticles can also be associated with an agent which targets selected cells and/or tissues to produce targeted contrast agents and/or targeted drug delivery agents.

Surfactant-stabilized microbubble mixtures for use as ultrasound contrast agents are disclosed in U.S. Pat. No. 5,352,436.

U.S. Pat. No. 5,637,289, U.S. Pat. No. 5,648,062, U.S. Pat. No. 5,827,502 and U.S. Pat. No. 5,614,169 disclose contrast agents comprising water-soluble, microbubble generating carbohydrate microparticles, admixed with at least 20% of a non-surface active, less water-soluble material, a surfactant or an amphiphillic organic acid. The agent is prepared by dry mixing, or by mixing solutions of components followed by evaporation and micronizing.

U.S. Pat. No. 6,139,819 discloses contrast agents for diagnostic and therapeutic uses comprising a lipid, a protein, polymer and/or surfactant, and a fluorinated gas, in combination with a targeting ligand. Such agents are particularly useful in imaging of an internal region of a patient suffering from an arrhythmic disorder.

U.S. Pat. No. 6,485,705 discloses imaging contrast agents useful in ultrasonic echography comprising gas or air filled microbubble suspensions in aqueous phases containing laminarized surfactants and, optionally, hydrophilic stabilizers. The laminarized surfactants can be in the form of liposomes. The suspensions are obtained by exposing the laminarized surfactants to air or a gas before or after admixing with an aqueous phase.

U.S. Pat. No. 6,375,931 discloses gas-containing contrast agent preparations for use in ultrasonic visualization of a subject, particularly perfusion in the myocardium and other tissues, which promote controllable and temporary growth of the gas phase in vivo following administration. Therefore, these agents act as deposited perfusion tracers. The preparations include a coadministerable composition comprising a diffusible component capable of inward diffusion into the dispersed gas phase to promote temporary growth thereof. In cardiac perfusion imaging, the preparations may be coadministered with vasodilator drugs such as adenosine in order to enhance the differences in return signal intensity from normal and hypoperfused myocardial tissue, respectively.

U.S. Pat. No. 6,524,552 discloses compositions of matter useful in imaging cardiovascular diseases and disorders. The compositions have the formula V—L—R where V is an organic group having binding affinity for an angiotensin II receptor site, L is a linker moiety or a bond, and R is a moiety detectable in in vivo imaging of a human or animal body.

U.S. Pat. No. 6,315,981 discloses a contrast medium for magnetic resonance imaging comprising gas filled liposomes prepared by a method wherein an aqueous suspension of a biocompatible lipid is agitated in the presence of a gas at a temperature below the gel to liquid crystalline phase transition temperature of the biocompatible lipid until gas filled liposomes result. The gas used in this contrast medium is hyperpolarized rubidium enriched xenon.

U.S. Pat. No. 6,264,917 discloses targetable diagnostic and/or therapeutically active agents, e.g. ultrasound contrast agents, having reporters comprising gas-filled microbubbles stabilized by monolayers of film-forming surfactants, the reporter being coupled or linked to at least one vector.

Lanzi et al. in U.S. Pat. No. 5,690,907, U.S. Pat. No. 5,958,371, U.S. Pat. No. 6,548,046 and U.S. Pat. No. 6,676,963 disclose lipid encapsulated particles useful in imaging by x-ray, ultrasound, magnetic resonance, positron emission tomography or nuclear imaging which comprise a molecular epitope on the surface of the particle for conjugation of a ligand thereto.

However, there remains a need for microcapsules and nanocapsules and methods of production of microcapsules and nanocapsules used for contrast imaging and/or drug delivery.

### BRIEF SUMMARY OF THE INVENTION

An object of the present is to provide a method for producing surfactant-stabilized microcapsules or nanocapsules including the steps of (a) preparing a suspension comprising a non-ionic sorbitan detergent and a salt in phosphate buffered saline; (b) adding to the suspension a nonionic polyoxyethylenesorbitan detergent to produce a solution; (c) heating while stirring the solution of step (b) to 55±5° C. and maintaining the temperature of the solution at 55±5° C. for several minutes; (d) allowing the solution to cool to room temperature; (e) autoclaving the solution; (f) creating surfactant-stabilized microbubbles and nanobubbles in the solution; and (g) collecting surfactant-stabilized nanocapsules and microcapsules formed from the microbubbles and nanobubbles.

Another object of the present invention is to provide methods for isolating nanobubbles or nanocapsules from mixed populations of microbubbles and nanobubbles or microcapsules and nanocapsules which comprises separating the nanobubbles or nanocapsules and collecting the lower layer following separation.

Another object of the present invention is to provide surfactant-stabilized microcapsules and nanocapsules produced in accordance with the methods of the present invention.

Another object of the present invention is to provide a contrast agent for diagnostic imaging in a subject which comprises surfactant-stabilized microcapsules and/or nanocapsules of the present invention that are filled with a gas. Such contrast agents may further comprise a targeting agent such as a peptide or antibody on the microcapsule and/or nanocapsule surface for targeting of the contrast agents to selected tissues or cells. Attachment of a targeting agent selective to a diseased tissue provides for a contrast agent which distinguishes between diseased and normal tissue. Use of contrast agents comprising the nanocapsules and/or microcapsules of the present invention permits imaging of tissues via access to locations of the vasculature too narrow for access via larger microcapsules, e.g. leaky tumor vasculature.

Another object of the present invention is to provide methods for imaging a tissue or tissues in a subject via administration of a contrast agent comprising surfactant-stabilized microcapsules and/or nanocapsules of the present invention that are filled with a gas. Contrast agents used in this method may further comprise a targeting agent such as a peptide or antibody on the microcapsule and/or nanocapsule surface for targeted delivery of the contrast agent to the selected tissue or tissues. Attachment of a targeting agent selective to a diseased tissue provides for a method of distinguishing via selective imaging diseased tissue from normal tissue. Similarly, attachment of a targeting agent selective to a malignant tissues provides for a method of distinguishing via selective imaging malignant tissue from benign tissue. Contrast agents of the present invention may be administered alone or in combination with additional agents including, but not limited to, free drug, genetic material, non-echogenic capsules with or without payload, or combinations thereof.

Another object of the present invention is to provide a composition for delivery of a bioactive agent which comprises a bioactive agent adsorbed to, attached to, and/or encapsulated in, or any combination thereof, surfactant-stabilized or polymer-based microcapsules and/or nanocapsules of the present invention. Such compositions may further comprise a targeting agent such as a peptide or antibody on the microcapsule and/or nanocapsule surface for targeting of the bioactive agent to selected tissues or cells. Attachment of a targeting agent selective to a diseased tissue provides for a delivery agent which delivers a bioactive agent selectively to diseased tissue. The bioactive agent can be released from the microcapsule and/or nanocapsule by exposure to an energy source such as ultrasound or other externally administered energy source and/or upon degradation of the surfactant-stabilized capsule. Use of compositions comprising the nanocapsules and/or microcapsules of the present invention permits delivery of bioactive agents to locations of the vasculature too narrow for access via larger microcapsules, e.g. leaky tumor vasculature. Compositions of the present invention may be administered alone or in combination with additional agents including, but not limited to, free drug, genetic material, non-echogenic capsules with or without payload, or combinations thereof.

Another object of the present invention is to provide methods for delivery of bioactive agents to a subject via administration of a composition comprising a surfactant-stabilized microcapsule and/or nanocapsule of the present invention and a bioactive agent adsorbed to, attached to, and/or encapsulated in, or any combination thereof, the surfactant-stabilized microcapsule and/or nanocapsule. Compositions used in this method may further comprise a targeting agent such as a peptide or antibody on the microcapsule and/or nanocapsule surface for targeting of the bioactive agent to selected tissues or cells in the subject. In this method, bioactive agent is released from the microcapsule and/or nanocapsule by exposure to ultrasound or other externally administered energy source, degradation of the surfactant-stabilized capsule or a combination thereof. Compositions of the present invention may be administered alone or in combination with an additional agent such as, but not limited to, free drug, genetic material, non-echogenic capsules with or without drug payload, or combinations thereof.

Yet another object of the present invention is to provide methods for enhancing delivery of a bioactive agent to selected tissues via vasculature and extravascular spaces too narrow for access by larger microcapsules which comprises administering to a subject a composition comprising the bioactive agent adsorbed to, attached to, and/or encapsulated in, or any combination thereof, a nanocapsule, preferably a surfactant-stabilized nanocapsule of the present invention, and exposing the subject to ultrasonic waves which force the composition through small leaks of the vasculature and extravascular spaces and extravascular spaces too narrow for access via large microcapsules by mechanisms including, but not limited to, cavitation and microstreaming. Enhancing delivery to a targeted tissue by ultrasound is useful in drug delivery techniques involving the present invention as well as imaging techniques.

### DETAILED DESCRIPTION OF THE INVENTION

The present invention provides surfactant-stabilized microcapsules and/or nanocapsules and methods for producing such microcapsules and nanocapsules which are useful as imaging agents and in drug delivery. The microcapsules and nanocapsules of the present invention can be modified to be loaded with bioactive agents. Further, the microcapsules and nanocapsules of the present invention can be modified on their surface with a bioactive moiety that specifically targets the microcapsule and/or nanocapsule to selected tissue types. Nanocapsules of the present invention, less than about 8 micrometers in diameter, more preferably less than about 700 nanometers in diameter, are capable of extravasation to specific tissues in areas such as a tumor and are capable of functioning as contrast agents in imaging techniques such as ultrasound. The nanocapsules and microcapsules of the present invention can also be used to carry and deliver a drug payload to a specific target in the body. Furthermore, these nanocapsules and microcapsules can be used to deliver the drug payload at a selected target through an energy triggering mechanism such as ultrasound or an alternative external energy source and/or rate predetermined biodegradation.

Ultrasound can also be used to enhance delivery of nanocapsules such as those disclosed herein to selected tissues via holes in the vasculature and extravascular spaces too narrow for access by larger microcapsules, e.g. leaky tumor vasculature. In this method, a composition comprising the bioactive agent adsorbed to, attached to, and/or encapsulated in, or any combination thereof, a nanocapsule, preferably a surfactant-stabilized nanocapsule of the present invention is administered to the subject. The subject can then be exposed to ultrasonic waves which force the composition through small holes of the vasculature and extravascular space too narrow for access by large microcapsules via mechanisms including, but not limited to, cavitation and microstreaming. Enhancing delivery to a targeted tissue by ultrasound is useful in drug delivery techniques involving the present invention as well as imaging techniques.

The surfactant-stabilized nanocapsules and microcapsules of the present invention are produced as follows. A non-ionic biological detergent, preferably a sorbitan, more preferably a Span such as Span 60 is crushed with a salt such as NaCl in a ratio by weight of greater than 1:10. Phosphate buffered saline (PBS) is then added in an amount sufficient to form a paste. Additional PBS is then added, preferably in a drop-wise fashion, in an amount sufficient to form a suspension. The suspension is then poured into beaker and rinsed with additional PBS. A solution comprising a second nonionic detergent, preferably a polyoxyethylenesorbitan, more preferably a Tween such as Tween 80 or modified Tween-PEG is then added to the suspension and the mixture is rinsed with 30 ml PBS. The resulting solution is stirred while being heated to approximately 55±5° C. The temperature of the solution is maintained at approximately 55±5° C. for several minutes, preferably 3 minutes, after which the solution is allowed to cool to room temperature. After cooling, the solution is autoclaved. Surfactant-stabilized micro-sized bubbles are then created in the solution either via sonication homogenization, or another method of creating a high shear in the fluid, or a combination thereof.

For sonication, a beaker of the solution is placed into a water bath at approximately 10° C. A biocompatible non-toxic gas, preferably air, PFC or SF₆, gas, is bubbled through a nozzle, purging the solution at approximately 40 ml/minute flow rate. The solution is then probe sonicated, preferably at ˜110 W for approximately 3 minutes while maintaining the bubbling of the gas.

Alternatively, homogenization can be used to create the surfactant-stabilized micro-sized agent. For homogenization, a beaker of the solution is placed onto a homogenizer with a saw-tooth blade. A biocompatible non-toxic gas, preferably air, PFC or SF₆, is then bubbled into the mixture through a nozzle, purging the solution preferably at approximately 40 ml/minute flow rate. Homogenization, preferably at approximately 12,000 RPM is continued for approximately 8 minutes while continuing gas flow.

Following sonication and/or homogenization, the solution is permitted to separate in a separation funnel into three layers. The middle layer contains the surfactant-stabilized microcapsules and nanocapsules of the present invention.

Alternatively, collection of the nanocapsules can be timed so that larger micron sized bubbles are collected first, drawn off, and the nano-sized bubbles are collected subsequently.

Modified Tween-PEG for use in production of the above nanocapsules and microcapsules can be prepared as follows. PEG is added to a CAA (chloroacetic acid)/sodium hydroxide solution and the reaction mixture is stirred for at least one hour. Following stirring, the reaction is stopped by addition of NaH₂PO₄, and the pH is adjusted to neutral. Excess reactants are removed, preferably by dialysis and the remaining solution is freeze dried to form PEG-dicarboxylate. The PEG-dicarboxylate is then added to a solution of Tween, preferably Tween 80 in MeCl₂ followed by addition of DCC(N,N′-dicyclohexylcarbodiimide). The resulting mixture is reacted with stirring for approximately 3 hours. Following this reaction, approximately half of the MeCl₂ is evaporated and the remaining solution is chilled in a cold room for 3-4 hours during which time a precipitate forms. This precipitate is filtered off and any remaining MeCl₂ is evaporated to obtain the modified Tween-PEG. The modified Tween-PEG can then be further modified with a targeting ligand using chemistry known to those skilled in the art, such as, but not limited to, carboiimide, either before its use in the bubble manufacture, or after bubbles are created.

Further, it has been found that nanocapsules with mean diameters ranging from about 700 nanometers to about 450 nanometers can be generated depending upon process variables. In addition, employing a centrifugation step allows for separation of these nano-sized capsules. Separation of the nanobubbles or nanocapsules can also be acheived by other separation techniques such as settling by gravity, or other methods of separation due to size, density or chemical properties known to those skilled in the art. For example, it has been found that the above prepared solution of surfactant stabilized microcapsules and nanocapsules will separate into layers based upon size change with an upper layer or region containing mostly microbubbles, and a lower layer or region containing suspended nanobubbles in buffer upon centrifugation for 1 minute at 500 RPM, centrifugation at 1 minute at 300 RPM or centrifugation for 3 minutes at 300 RPM. Mean diameter of bubbles in the lower layer following centrifugation for 1 minute at 300 RPM was 0.69 micrometer, while means size after 3 minutes at 300 RPM or 3 minutes at 500 RPM were 0.45 micrometers and 0.49 micrometers respectively. Thus, the population of nanocapsules of the present invention, particularly useful in targeting selected tissues via narrow vasculature and extravascular space, can be enhanced via a size separation technique such as centrifugation and collection of the lower layer.

Using in vitro acoustic measurement, dose response curves at 5 MHz insonation were generated for each of these samples subjected to different centrifugation conditions. Differences in the dose response curves were observed for each. These differences are believed to be influenced by the nanobubble concentrations. Relevant test doses placed into the acoustic chamber containing 750 ml of PBS buffer ranged from 10 to 150 μl for the 1 minute, 300 RPM samples, 25 to 500 μl for the 3 minute, 300 RPM samples and from 100 to 1500 μl for the 3 minute, 500 RPM samples. Maximum enhancement was found to be around 27 dB at a dose of 50 μl for the 1 minute, 300 RPM samples, 25.5 dB at a dose of about 200 μl for the 3 minute, 300 RPM samples, and 24 dB at a dose of 500 μl for the 3 minute, 500 RPM samples. Thus, it appears that as centrifugal force and time increases, the maximum enhancement decreases and the agent becomes more dilute.

Time response curves under insonation at 5 MHz were also determined based upon results of the dose response curves. An initial dose of 25 μl was chosen for the 1 minute, 300 RPM samples, 75 μl for the 3 minute, 300 RPM samples, and 500 μl for the 3 minute, 500 RPM samples. The 1 minute, 300 RPM sample remained relatively stable throughout the 15 minute insonation period, with an acoustic drop of less than 5 dB. The 3 minute, 300 RPM samples remained stable through 8 minutes, with a mean acoustic drop of less than 5 dB, followed by a more rapid acoustic drop-off. The 3 minute, 500 RPM samples were the least stable, having an acoustic drop of more than 5 dB after 6 minutes.

As will be understood by those skilled in the art upon reading this disclosure, similar separation techniques may be applicable to isolate nanocapsule populations of other contrast agents useful in imaging techniques such as X-ray, MRI and PET.

Samples of the contrast agent of the present invention injected into New Zealand white rabbit via a catheterized ear vein showed significant enhancement at an injected dose of 0.1 ml/kg. Under power Doppler, using 12.5 Mhz transducer with a pulse repetition frequency (PRF)=700 Hz and mechanical index (MI)=0.33, clinically significant enhancement lasted 1 minute 29 seconds as judged by a trained sonographer. Using pulse inversion harmonic imaging, an L7-4 tranducer with a PRF=700 Hz and mechanical index MI=0.26, strong and clinically significant enhancement lasted 1 minute 58 seconds as judged by a trained sonographer.

The surfactant-stabilized microcapsules or nanocapsules of the present invention can also be loaded with a bioactive compound. Examples of bioactive agents which can be adsorbed, attached and/or encapsulated in the microcapsules and/or nanocapsules of the present invention include, but are not limited to, antineoplastic and anticancer agents such as azacitidine, cytarabine, fluorouracil, mercaptopurine, methotrexate, thioguanine, bleomycin peptide antibiotics, podophyllin alkaloids such as etoposide, VP-16, teniposide, and VM-26, plant alkaloids such as vincristine, vinblastin and paclitaxel, alkylating agents such as busulfan, cyclophosphamide, mechlorethamine, melphanlan, and thiotepa, antibiotics such as dactinomycin, daunorubicin, plicamycin and mitomycin, cisplatin and nitrosoureases such as BCNU, CCNU and methyl-CCNU, anti-VEGF molecules, gene therapy vectors and other genetic materials and peptide inhibitors such as, but not limited to, MMP-2 and MMP-9, which when localized to tumors prevent tumor growth.

The microcapsules and/or nanocapsules of the present invention may further comprise a targeting agent attached to the capsule surface, which upon systemic administration can target the contrast agent or the delivery agent to a selected tissue or tissues, or cell in the body. Targeting agents useful in the present invention may comprise peptides, antibodies, antibody fragments, or cell surface receptor-specific ligands that are selective for a tissue or cell. Examples include, but are in no way limited to, RGD which binds to αv-integrin on tumor blood vessels, NGR motifs which bind to aminopeptidase N on tumor blood vessels and ScFvc which binds to the EBD domain of fibronectin. Accordingly, targeting agents can be routinely selected so that a contrast agent or delivery agent of the present invention, or a combination thereof, is directed to a desired location in the body such as selected tissue or tissue, cells or an organ, or so that the contrast agent or delivery agent of the present invention can distinguish between various tissues such as diseased tissue versus normal tissue or malignant tissue versus benign tissue. Targeted contrast and/or delivery agents can be administered alone or with populations of contrast agents and/or delivery agents of the present invention which do not further comprise a targeting agent.

Surface-modified, gas-filled surfactant-based nanocapsules and microcapsules that are made according to the above method are useful in medical applications such as targeted imaging contrast agents for cancer or tissue perfusion because their size allows them to penetrate into most any tissue. Further penetration of the nanocapsules can be enhanced by ultrasonic waves which force the nanocapsules through leaks of the vasculature and extravascular spaces and extravascular space via mechanisms including, but not limited to, cavitation and microstreaming and to their target tissues. For example, the ultrasonic wave can be tuned to interact with the contrast agent in a manner which causes cavitation or microstreaming, both of which will aid in displacing the agent or contents thereof through gap junctions in the capillaries. The ultrasound beam is preferably focused on the area of interest, for example, the targeted tissue for delivery such as a tumor. The nanocapsules can also be injected into the vascular system for parenteral administration, into or close to the lymphatic system or directly into a tumor or organ for local delivery. The drug/bioactive payload can also be used to stimulate angiogenesis in situations where this is advantageous such as tissue engineering constructs and replacement implants in areas such as the hip and damaged hearts and other wound repair. Release of the drug can be triggered by administration of an energy source such as ultrasound. Release can also be triggered by degradation of the microcapsule or nanocapsule. Degradation of the microcapsules and nanocapsules and release of the drug can be modified by applying outside energy forces such as ultrasound or heat. Accordingly, these nanocapsules and microcapsules of the present invention are useful in targeted ultrasonic imaging, targeted ultrasonic drug delivery, cancer diagnosis, cancer detection, prostate evaluation, and evaluation of angiogenesis for implants.

Nanocapsules of the present invention may be administered alone or in combination with additional agents including, but not limited to, free drug, genetic material, non-echogenic capsules with or without payload, or combinations thereof.

The following nonlimiting examples are provided to further illustrate the present invention.

### EXAMPLES

### Example 1

### Capsule Fabrication of Nanobubble Surfactant-Based Ultrasound Contrast Agent

Span 60 (1.48 grams) and NaCl (12.50 grams) were crushed with a mortar and pestle. Phosphate buffered saline (PBS; 3 ml) was added and the mixture was crushed to form a paste. An additional 7 ml of PBS was added in a drop-wise fashion to form a suspension. The suspension was then poured into beaker and rinsed with 10 ml PBS.

Tween 80 (1 ml) or modified Tween-PEG (1 gram) was then crushed with a mortar and pestle. A total of 10 ml PBS was added to form solution. This solution was then added to the suspension of Span 60 and NaCl, followed by rinsing with 30 ml PBS.

The solution was stirred and then heated to 55±5° C. The solution was held at 55±5° C. for 3 minutes and then allowed to cool to room temperature.

After cooling, the solution was autoclaved using a liquid cycle at 120° C. for 12 minutes. Sonication of the resulting solution created surfactant-stabilized micro-sized bubbles. For sonication, 50 ml of the above solution was drawn into a 150 ml beaker. The beaker was placed into a water bath (˜10° C.). The desired gas, preferably PFC or SF₆ was bubbled through a nozzle, purging the solution at ˜40 ml a minute flow rate. The solution was then probe sonicated at ˜110 W for 3 minutes while maintaining the bubbling of the gas (˜30 to 40 ml/minute) just below the sonicator tip. Once the sonication was stopped, the gas was also stopped.

Alternatively, homogenization was used instead of sonication to create the surfactant-stabilized micro-sized agent. Use of homogenization decreased the yield. For homogenization, 50 ml of the solution (mixture of Span 60 and Tween 80 prepared as described above) was drawn into a 150 ml beaker. The mixture was then placed onto a homogenizer with a saw-tooth blade. The blade was positioned close to bottom of the beaker (˜1-2 mm). The desired gas, preferably PFC or SF₆ was then bubbled into the mixture through a nozzle, purging the solution at ˜40 ml per minute flow rate. Homogenization was at 12,000 RPM for 8 minutes while continuing gas flow. After finishing homogenization, the gas was turned off.

Unreacted solutions were then removed by first collecting the entire micron and submicron population of agent, then separating the nano-sized agent. The solution was decanted into a 250 ml separating funnel, inside a cold-room (˜15° C.). PBS (60 ml) was used to wash the solution inside the separating funnel. The solution was allowed to separate over approximately 35 minutes into three layers. Approximately 95% of the bottom layer was bled off followed by addition of a second 60 ml aliquot of PBS to the separation funnel using a small pipette, thus washing the bubbles. The solution was again allowed to separate for approximately 35 minutes into three layers. This washing procedure was repeated 3 times. After the final washing step, the solution was allowed to separate into three layers for approximately 35 minutes. The clear lower layer was decanted off and the middle layer containing micro-bubbles was collected into a beaker.

The collected microbubbles were then diluted with fresh PBS (1:1 ratio) and mixed to prevent premature separation. The mixture was centrifuged at 500 RPM for 3 minutes and the bottom layer (nano-bubbles) of solution was collected into 20 ml glass disposable scintillation vials. The top of each vial was purged with the gas, capped and sealed using parafilm. Vials were stored in the cold room.

### Example 2

### Separation of Microbubbles and Nano-Bubbles

The microbubble solution of Example 1 was transferred to a 50 ml centrifuge tube equipped with a drainage port at the base. Suspended microbubbles were centrifuged (Beckman Coulter Allegra 21, rotor S4180) for one of the following: 1 minute at 500 RPM (RCF 45), 1 minute at 300 RPM (RCF 16) or 3 minutes at 300 RPM. In most cases the solution separated into two distinct layers: an upper layer containing mostly bubbles, and a lower layer containing suspended bubbles in buffer. The liquid (lower) layer of the solution was collected for size and acoustic analysis, and the top layer was discarded. If no distinct layer was observed, 7.5 ml (consistent with the layered samples) was collected from the bottom of the centrifuge tube. Collected agent was purged with PFC gas and stored at 4° C. in tightly capped vials sealed with parafilm.

### Example 3

### Size Analysis

The mean diameter size of the bubbles was analyzed using a Horiba LA-910 laser scattering particle size analyzer. The relative refractive index (RRI) setting chosen was 1.00+1.00i, based on results of refractive measurements done with ST68 and PBS. The real part indicates the refraction of light relative to water, and the imaginary part indicates the amount of light absorbed by the sample. The particle size distribution was determined by using the length algorithm. PBS was used as the blank solution, an agent was added until an appropriate concentration was indicated by the Horiba. Each sample was tested in triplicate.

### Example 4

### Acoustic Measurements

Acoustic measurements were conducted on the centrifuged agent. The agent was added to 0.75 liters of PBS solution in a plexiglass vessel equipped with an acoustic window submerged in a deionized water tank and analyzed using a custom LabVIEW (National Instruments) program interfaced with an oscilloscope as described by Raisinghani and DeMaria (American Journal Cardiology 2002 90:3J-7J). Acoustic measurements were conducted with a one-dimensional pulsed A-mode ultrasound set-up with interchangeable single broadband, 12.7 mm element diameter, 508 mm spherically focused transducer with a center frequency of 5 MHz (Panametrics, Inc.). The −6 dB bandwidth of the transducer was 92%. The transducer was inserted into a degassed, deionized water bath (25° C.) and focused through an acoustic window of the sample vessel. A pulser/receiver (model 5072PR, Panametrics, Inc.) was used to pulse the transducer at a pulse repetition frequency of 100 Hz. The received signals were amplified to 40 dB and fed to a digital oscilloscope (Lecroy 9350A), which were then sent to a computer, stored and analyzed using LabView. The reference (PBS) was taken as an average of 6 values.

Dose response curves were constructed (dose in μl vs. enhancement in dB) for doses in the range of 10-1500 μl depending on the sample. Three samples per preparation were tested, and at least 5 measurements per curve for each sample were obtained. Time responses were conducted with the same acoustic setup for 15 minutes with a starting dose based on the dose response curve results.

### Example 5

### Preparation of Modified Surfactant

### Modified Tween-PEG

NaOH (3M), and HCl (6N) solutions were prepared. A 0.5 M CAA(chloroacetic acid) solution in 100 ml of NaOH solution was also prepared. PEG (5 grams) was added to the CAA(chloroacetic acid) solution and stirred for 70 minutes. Following stirring, the reaction was stopped by adding 1 gram of NaH₂PO₄. The pH of the reaction mixture was then adjusted to neutral with HCl. Excess reactants in the mixture were then removed by dialysis and the remaining solution was freeze dried to form PEG-dicarboxylate.

Tween 80 (2 ml) was dissolved in 25 ml of MeCl₂. PEG-dicarboxylate (1 gram) was then added followed by 50 mg DCC(N,N′-dicyclohexylcarbodiimide). The mixture was allowed to react with stirring for 3 hours. Following the reaction, approximately half of the MeCl₂ was evaporated and the remaining reaction mixture was placed in cold room for approximately 3-4 hours. Following cooling the resulting precipitate was filtered off and remaining MeCl₂ was evaporated.

### Example 6

### Conjugation of Peptide to Surfactant-Stabilized Contrast Agent

Modified bubbles of Example 1 were combined with 5.0 mg EDC (˜1:1 molar ratio of COOH groups on bubbles to EDC), 2.7 mg NHS (1:2 molar ratio to EDC), and 10 ml of buffer (0.1M MES, 0.3M NaCl, pH 6.5) and shaken on a shaker for 15 minutes. A peptide GRGDS (150 g, ˜1:10 molar ratio of COOH groups on bubbles to peptide) was then added and shaken for 3 hours. Conjugated bubbles were then washed using a 250 ml separating funnel and PBS (60 ml) and agent with the conjugated peptide was collected in 30 ml of PBS.

What is claimed is:

1. A method for producing surfactant-stabilized microcapsules or nanocapsules comprising:
(a) preparing a suspension comprising a non-ionic sorbitan detergent and a salt in phosphate buffered saline;
(b) adding to the suspension a nonionic polyoxyethylenesorbitan detergent to produce a solution;
(c) heating while stirring the solution of step (b) to 55±5° C. and maintaining the temperature of the solution at 55±5° C. for several minutes;
(d) allowing the solution to cool to room temperature;
(e) autoclaving the solution;
(f) creating surfactant-stabilized microbubbles and nanobubbles in the solution; and
(g) collecting surfactant-stabilized nanocapsules and microcapsules formed from the microbubbles and nanobubbles.

2. The method of claim 1 further comprising separating the surfactant-stabilized nanocapsules from the surfactant-stabilized microcapsules into a lower layer or region as compared to the surfactant-stabilized microcapsules and collecting the lower layer or region of isolated nanocapsules.
